(19) United States
(12) Plant Patent Application Publication
(10) Pub. No.: US 2026/0060158 P1
(43) Pub. Date: Feb. 26, 2026

(54) MUSHROOM VARIETY 'RO12756'

(50) ;

(71) Applicant: Amycel, LLC, Watsonville, CA (US)

(72) Inventors: Christopher W. ROBLES, Watsonville, CA (US); Maykoe R. ABAUNZA, Memphis, TN (US)

(73) Assignee: Amycel, LLC, Watsonville, CA (US)

(21) Appl. No.: 19/305,683

(22) Filed: Aug. 20, 2025

Related U.S. Application Data
(60) Provisional application No. 63/685,196, filed on Aug. 20, 2024.
(60) Provisional application No. 63/733,289, filed on Dec. 12, 2024.

(51) Int. Cl. A01H 15/00

(52) U.S. Cl. CPC A01H 15/00
USPC PLT/394

(57) ABSTRACT

A new and unique brown variety of the mushroom Agaricus bisporus Imbach was produced by crossing the progeny of brown mushrooms and off-white hybrid mushrooms to bridging cross strains. The resultant hybrid, named ‘RO12756’, exhibits distinct and stable characteristics. Hybrid ‘RO12756’ exhibits a light cap color, thicker cap flesh, rounder cap, faster mushroom development, and high yield, especially when compared to available brown mushroom varieties.

### CROSS-REFERENCE TO RELATED APPLICATIONS

This application claims the benefit of U.S. Provisional Application No. 63/685, 196, filed Aug. 20, 2024, and U.S. Provisional Application No. 63/733,289, filed Dec. 12, 2024, which are hereby incorporated by reference in their entirety.

Latin name: Genus and species: The mushroom cultivars of this disclosure are identified as Agaricus bisporus Imbach.

Varietal denomination: Variety denomination: The variety denomination is ‘RO12756’.

### BACKGROUND OF THE INVENTION

The cultivated white button variety of Agaricus bisporus, known as A. bisporus (Lange) Imbach (syn. A. brunnescens Peck), is the predominant mushroom species in cultivation in the world today. After many years in which commercial mushroom sales in the United States were restricted primarily to white-capped A. bisporus mushrooms, there has been a recent trend toward increased sales of brown-capped A. bisporus mushrooms of various types and other so-called exotic strains (species other than A. bisporus). Improved agronomic characteristics can be introduced into the hybrid strains that can influence producer profitability. For instance, strains may have different ancestry, which will be reflected directly by the genotype and indirectly, in some cases, by the phenotypical characteristics. Generally speaking, strains may be differentiated on the basis of traits associated with the hybrid strains, such as, but not limited to, cap shape, surface texture, flesh thickness, color, shelf life, basidial spore number, sporelessness, resistance to infection by, symptoms of, or transmission of bacterial, viral or fungal diseases, amongst many other traits, to give some examples Many of these traits directly contribute to factors that influence productivity, revenue capture, reduced costs, crop performance, and consumer and market demand.

In the endeavor to produce new mushroom varieties possessing desirable and improved characteristics, a preferred method is the crossing of homokaryons, ‘homokaryons’ being a multinucleate haploid fungal culture possessing a single line (somatic lineage) that is capable of undergoing plasmogamy with one or more other homokaryons. Each genetically different homokaryon is representative of a parent line in a resultant heterokaryon. The degree of success of each strain and the profitability of producers can be improved by the appropriate selection of the parental lines. Additionally, improved visual appeal and improved shelf-life, amongst many other characteristics, can also contribute to increased sales for mushroom growers in the world today. Thus, there are many characteristics by which a novel strain might be judged as superior in a particular production facility or a sales market, regionally or globally.

It is also well understood that an industry relying extensively only on a single genetic lineage is at increased risk of unpredictable, catastrophic crop failure on a worldwide scale due to the spread of diseases. To alleviate this type of problem, development and cultivation of new hybrid strains with independent lineages will help to act as a disease break. Further, it is highly desirable to simultaneously provide both genetic diversification and commercially acceptable improved crop characteristics. Thus, the need continues to exist for new brown hybrid strains of Agaricus bisporus with improved and preferred characteristics.

### BRIEF SUMMARY OF THE INVENTION

It is an object of the disclosure to provide a brown variety of Agaricus bisporus mushroom with improved commercial characteristics relative to existing brown commercial mushrooms. Strains of the new brown variety, as well as qualities of this new variety, are described herein.

The following traits have been repeatedly observed and are determined to be the unique characteristics of the novel brown Agaricus bisporus mushroom strains. Specifically, it is an object of the disclosure to provide mushrooms that have one or more (most preferably all) of these following characteristics (relative to brown Agaricus bisporus now being marketed).

These characteristics in combination distinguish ‘RO12756’ as a new and distinct brown variety:
1. Faster or as fast timing to reach pinset, first break, and second break as marketed brown varieties.
2. High yield, especially during the second and third break.
3. Cap apex flesh thicker than marketed brown varieties.
4. Cap edge flesh thicker than marketed brown varieties.
5. Soft cap firmness.
6. Light brown cap color.
8. Stipes more wide or as wide as marketed brown varieties.
9. Higher or as high tray coverage as marketed brown varieties.
10. Incompatibility with other available brown strains.

These and other objects of the disclosure have been accomplished by providing a hybrid Agaricus bisporus mushroom strain obtained by crossing a mushroom of wild strain AA-0096 or a progeny thereof with a second Agaricus bisporus strain comprising a commercial off-white hybrid strain, wherein the mushroom of the disclosure (1) has (a) at least one genetic characteristic of wild strain AA-0096 not present in the second Agaricus bisporus strain, (b) at least one genetic characteristic of the second Agaricus bisporus strain not present in wild strain AA-0096, and (c) at least one genetic characteristic of the bridging cross strain and (2) has at least one physical characteristic selected from the group consisting of pinset speed, cap color, cap apex flesh thickness, cap edge flesh thickness, cap texture, tray coverage, incompatibility with other strains, speed of harvestable yield in each break, amount of yield in each break, and overall yield that is statistically better than the corresponding physical characteristic of comparison strains ‘BR06’ or ‘B14528’.

Another feature of the present disclosure includes a new brown hybrid mushroom having improved agronomic traits leading favorably to increased consumer and market demand, productivity, crop performance, and revenue. Of particular interest and novel advantage are the following traits exhibited by ‘RO12756’:
1. Higher overall yield, driven by increased yield at later breaks, which enable growers to achieve higher yield per unit area compared to other commercial brown mushrooms.
2. Faster developmental timing, which enables growers to increase their efficiency by allowing them to pick off mushrooms before the end of the growing cycle. Faster strains might also allow for more crops per year. Traits herein related to developmental timing include Days to Pinset, Days to 1st Break, Days to 2nd Break, and Percent Harvested Speed.
3. Thick-fleshed morphology, which reflects higher quality than other commercial brown mushrooms. This morphology remains more consistent at later breaks for ‘RO12756’ when compared to other commercial varieties, and this increases visual appeal of sliced product. Pre-sliced button mushrooms have been growing in popularity over the last 10+ years; a 10-year report on mushroom sales trends demonstrated that sliced product has eclipsed whole product fresh mushroom sales, encompassing over 50% of fresh brown mushrooms sold in 2023 in the United States (Roerink, A. (2023 October). Ten-year Trends in Fresh Mushroom Sales. Mushroom Council. Available at www[dot]mushroomcouncil[dot]org/wp-content/uploads/2 023/10/Ten-Year-Fresh-Mushroom-Trends[dot]pdf).

A further feature of the present disclosure is a novel breeding pedigree having beneficial characteristics unique to the new hybrid strain differentiating it from other commercial brown strains.

### BRIEF DESCRIPTION OF THE DRAWINGS

The patent or application file contains at least one drawing executed in color. Copies of this patent or patent application publication with color drawings will be provided by the office upon request and payment of the necessary fee.

FIG. 1 shows a representative comparison of Agaricus bisporus variety ‘BR06’ (left) and variety ‘RO12756’ (right) tray coverage at first break. ‘BR06’ displays lower tray coverage, and ‘RO12756’ displays higher tray coverage.

FIG. 2 shows a longitudinal slice of a mushroom of variety ‘RO14176’ with markings added to illustrate the calculation of Cap Edge Flesh Thickness, where Cap Edge Flesh Thickness is calculated by ((LG_{L}−CS_{L})+(CS_{R}−RG_{R}))/CS_{D}. LG_{L}=left-most x-coordinate of the left gill, CS_{L}=left-most x-coordinate of the cross-section, CS_{R}=right-most x-coordinate of the cross-section, RG_{R}=right-most x-coordinate of the right gill, and CS_{D}=diameter of the cross-section.

FIG. 3 shows a longitudinal slice of a mushroom of variety ‘RO14176’ with markings added to illustrate the calculation of Cap Apex Flesh Thickness, where Cap Apex Flesh Thickness is calculated by (CS_{T}−((LG_{T}+RG_{T})/2))/CS_{H}. CS_{T}=the top-most y-coordinate of the cross-section, LG_{T}=the top-most y-coordinate of the left gill, RG_{T}=the top-most y-coordinate of the right gill, and CS_{H}=the height of the cross-section.

FIG. 4 shows a representative comparison of cap colors for variety ‘BR06’ (left), variety ‘B14528’ (center), and variety ‘RO12756’ (right) cap colors based on Cap L* Value, Cap a* Value, and Cap b* Value at first break. Mushrooms pictured were grown in the same room, under the same environmental conditions, and were harvested from the same trial in May 2025. All photos have the same scale. Scale bar=1 cm.

FIG. 5 shows representative comparisons of cap height, relative cap height, and relative cap flesh thickness for variety ‘B14528’ (left), variety ‘BR06’ (center), and variety ‘RO12756’ (right) at first break. Mushrooms pictured were grown in the same room, under the same environmental conditions, and were harvested from the same trial in May 2025. All photos have the same scale. Scale bar=1 cm.

### DETAILED BOTANICAL DESCRIPTION

In mushroom breeding, mycelial (=vegetative) cultures of two compatible progenitors (typically these are haploid homokaryotic strains called homokaryons) must come into physical contact so that one or more fusion zones can occur between the progenitors. Within those fusion zones nuclei from the two progenitors become associated. In Agaricus bisporus, a novel, hybrid mycelium ultimately containing two compatible haploid nuclear types (one from each of the two progenitors) plus one mitochondrial type (from either one of the progenitors) emerges. This novel hybrid mycelium can be isolated and propagated to provide the new hybrid culture, which can be further subdivided and propagated for commercial or other purposes.

It should be recognized that the mushrooms of the present disclosure are hybrids (equivalent to crosses), as they are formed by the hybridization of at least two independently derived strains, since they were formed by hybridizing the wild strain AA-0096 with a second, commercialized strain of A. bisporus (with the second strain, in some embodiments, being itself a cross between at least two other identified strains of A. bisporus). The term ‘hybrid mushroom’ may be used herein to represent the product of a cross between a wild A. bisporus strain and a commercial strain, often including a cross between a brown mushroom strain and an off-white hybrid variety. Differences in terminology (and in the specific techniques used alongside the terminology) are merely indicative of the variety of terminology and techniques used in the mushroom production field.

After parental homokaryotic spores germinated into mycelia, single spore isolates (SSIs) were selected, preferentially those exhibiting slow mycelial growth. The slowly growing mycelia of SSIs usually contain one nuclear type, are thus homokaryotic, and are suitable for crossing, and thus for breeding. Slow growers are denoted as “sg”. Some homokaryons were obtained through protoplasting; this technique involves treating the vegetative mycelia of the heterokaryons to damage the fungal cell wall, then selecting colonies of protoclones that are each derived from a single, regenerated protoplast. All crosses were initially cultivated on a small scale, and spores were collected for the parents of the crosses to produce the hybrids described herein.

One useful feature in the breeding programs for these novel mushroom varieties is the use of a bridging cross strain, which is a hybrid variety produced from crossing a commercial brown variety with a commercial white variety. The particular bridging cross strain of note in these breeding programs is known as the 4x29 strain. Strain 4x29 has been previously deposited under the provisions of the Budapest Treaty with the American Type Culture Collection, Rockville, Md., USA, ATCC accession No. PTA-6877 (deposited on Jul. 20, 2005). Strain 4x29 is the product of crossing a commercial brown strain with a commercial white strain. The ‘29’ of this strain is a unique homokaryon collected from an off-white hybrid strain.

The varieties, mushrooms, strains, and cultures disclosed herein are understood to comprise parts of mushrooms, such as the fruiting body, cap, stipe, basidium, spore, mycelium, mixed or unmixed spawn, inoculated substrate, protoplast, gill, cell, or product produced from the mushroom. In some embodiments, the mushroom part from the hybrid mushroom produced by any method disclosed herein is a spore. In some embodiments, the mushroom part produced from a mushroom strain disclosed herein is a spore.

### Trait Determinations

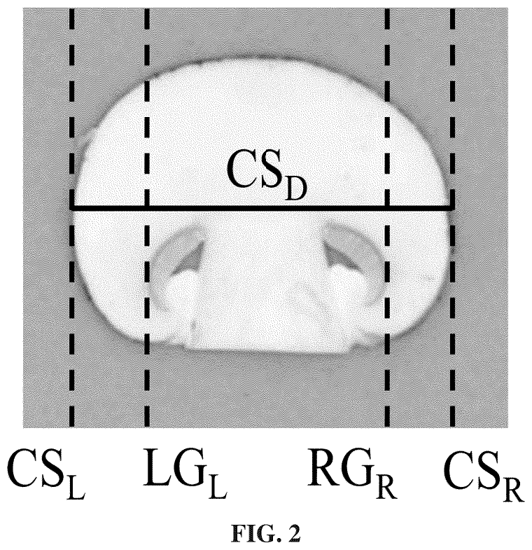

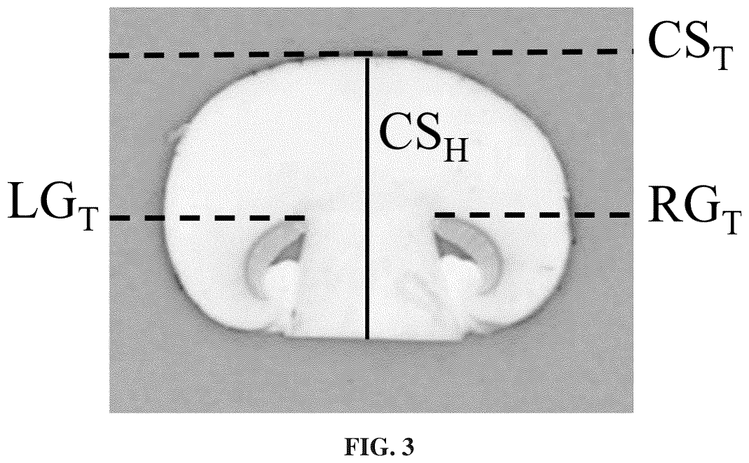

In the descriptions and tables that follow, numerous traits are calculated/measured as follows in Table 1. Visual depictions of the measurements used for calculating Cap Apex Flesh Thickness are shown in FIG. 3. Visual depictions of the measurements used for calculating Cap Edge Flesh Thickness are shown in FIG. 2.

Mushrooms were harvested at Stage 3 as described by Hammond & Nichols (Hammond, J. B. W., & Nichols, R. (1976). Carbohydrate metabolism in Agaricus bisporus (Lange) Sing.: changes in soluble carbohydrates during growth of mycelium and sporophore. Microbiology, 93(2), 309-320), when cap diameters were approx. 3-5 cm with the veil still intact. To reduce confounding effects introduced by different trials, developmental timing and yield traits were compared only from trials where all strains being compared were present together.

Images of mushroom caps and longitudinal cross-sections were captured using a Canon Rebel SL1 camera (Canon, Inc., Toyko, Japan) in a custom-built photo box with standardized lighting conditions. The camera was calibrated for color consistency using a ColorChecker gray balance card (Calibrite LLC, Wilmington, Del.). L*, a* and b* values were captured across the surface of each mushroom cap and cross-section using ImageJ (Schneider, C. A., Rasband, W. S., & Eliceiri, K. W. (2012). NIH Image to ImageJ: 25 years of image analysis. Nature Methods, 9(7), 671-675). Images captured in 2024 and 2025 were used for analysis. Extreme outliers were excluded from the data, defined as those falling 3 times the interquartile range (IQR) above the 75th percentile for that trait or more than 3 times the IQR below the 25th percentile for that trait.

All statistical analyses were carried out in the R programming language (R Core Team, (2013). R: A language and environment for statistical computing. R Foundation for Statistical Computing, Vienna, Austria. www[dot]R-project[dot]org/). Levene's test was performed using the ‘car’ package (Fox, J., & Weisberg, S. (2018). An R companion to applied regression. SAGE Publications), Shapiro-Wilk tests and Tukey HSD were performed using the ‘stats’ package (R Core Team (2013)), and standard ANOVAs, Kruskal-Wallis tests and Dunn's tests were performed using the ‘rstatix’ package (Kassambara, A (2023). rstatix: Pipe-Friendly Framework for Basic Statistical Tests. R package version 0.7.2, rpkgs[dot]datanovia[dot]com/rstatix/).

TABLE 1

| Trait | Tool | Measurement Scale | Timing |
|---|---|---|---|
| Cap Color Darkness | visually | 0 to 5 (from white to dark brown), | 1st break**, |
| | assessed | tray-level measurement | 2nd break** |
| Cap Color a* Value | image analysis | −128 to +127 (from pure green to pure | 1st break**, |
| (Green-Red) | | red), individual mushroom-level | 2nd break** |
| | | measurement | |
| Cap Color b* Value | image analysis | −128 to +127 (from pure blue to pure | 1st break**, |
| (Blue-Yellow) | | yellow), individual mushroom-level | 2nd break** |
| | | measurement | |
| Cap Color L* Value | image analysis | 0 to 100 (from pure black to pure white), | 1st break**, |
| (Dark-Light) | | individual mushroom-level measurement | 2nd break** |
| Cap Diameter | image analysis | millimeters (mm), individual mushroom- | 1st break**, |
| | | level measurement | 2nd break** |
| Cap Firmness | manual | 1 to 5 (from very soft to very firm), | 1st break**, |
| | squeeze test | tray-level measurement | 2nd break** |
| Cap Apex Flesh | calculated | distance from top of gills to top of cap of | 1st break**, |
| Thickness | ratio and | mushroom, relative to cap height, | 2nd break** |
| | image analysis | individual mushroom-level measurement | |
| Cap Edge Flesh | calculated | sum of distances from outer edge of each | 1st break**, |
| Thickness | ratio and | gill to outer edges of cap of mushroom, | 2nd break |
| | image analysis | relative to cap diameter, individual | |
| | | mushroom-level measurement | |
| Cap Flesh Thickness | hand- | millimeters (mm), distance from top of | 1st break**, |
| | measurement | gill-stipe connection (at center of stipe) to | 2nd break** |
| | | top of cap, individual mushroom-level | |
| | | measurement | |
| Cap Height | image analysis | millimeters (mm), distance from base of | 1st break**, |
| | | cap to top of cap, individual mushroom- | 2nd break** |
| | | level measurement | |
| Cap Roundness | image analysis | Unitless ratio (0-1), | 1st break**, |
| (Calculated) | | calculated as 4 * CS_{A}/(π * CS_{D}²), where | 2nd break** |
| | | CS_{A} = area of the cross-section, and | |
| | | CS_{D} = diameter of the cross-section; | |
| | | assessed with image analysis, individual | |
| | | mushroom-level measurement | |
| Cap Roundness (Visual) | visually | 1 to 5 (from very flat to spherical), | 1st break**, |
| | assessed | tray-level measurement | 2nd break** |
| Compatibility | calculated | pounds per square foot (lb/ft²) and | 1st break** |
| | ratio | kilograms per square meter (kg/m²), | |
| | | weight of 1st break mushrooms yielded | |
| | | relative to area of tray, tray-level | |
| | | measurement | |
| Compost Colonization | visually | 0 to 5 (from no colonization to robust | before 1st break |
| | assessed | colonization), tray-level measurement | (10 days after |
| | | | spawning) |
| Days to 1st Break | visually | number of days elapsed after casing at | 1st break ** |
| | assessed | which at least half of tray is of harvestable | |
| | | maturity for 1st break, tray-level | |
| | | measurement | |
| Days to 2nd Break | visually | number of days elapsed after casing at | 2nd break** |
| | assessed | which at least half of tray is of harvestable | |
| | | maturity for 2nd break, tray-level | |
| | | measurement | |
| Days to Pinset | visually | number of days elapsed after casing at | before 1st break |
| | assessed | which pins first reach 5 mm, tray-level | |
| | | measurement | |
| Percent Harvested Speed | calculated | % of yield harvested by a designated date | 17 days after |
| | ratio | within break, | casing (1st break), |
| | | yield at date within break/total yield for | 24 days after |
| | | break, trial-level measurement | casing (2nd break), |
| | | | 32 days after |
| | | | casing (3rd break) |
| Relative Cap Flesh | calculated | Cap Flesh Thickness Value/Cap Diameter | 1st break**, |
| Thickness | ratio | Value, individual mushroom-level | 2nd break** |
| | | measurement | |
| Relative Cap Height | calculated | Cap Height Value/Cap Diameter Value, | 1st break**, |
| | ratio | individual mushroom-level measurement | 2nd break** |
| Relative Stipe Diameter | calculated | Stipe Diameter Value/Cap Diameter | 1st break**, |
| | ratio | Value, individual mushroom-level | 2nd break** |
| | | measurement | |
| Stipe Diameter | hand- | millimeters (mm), stipe diameter at base of | 1st break**, |
| | measurement | cap, individual mushroom-level | 2nd break** |
| | | measurement | |
| Tray Coverage | visually | 0 to 5 (from no coverage to full coverage), | 1st break**, |
| | assessed | tray-level measurement | 2nd break** |
| Yield (Each Break) | weighing | kg/m², tray-level measurement; or | Assessed |
| | | lb/ft², tray-level measurement | cumulatively |
| | | | throughout 1st, |
| | | | 2nd, and 3rd |
| | | | breaks |
| Yield (Total) | weighing | kg/m², tray-level measurement; | Assessed |
| | | or lb/ft², tray-level measurement | cumulatively |
| | | | across 1st, 2nd, |
| | | | and 3rd breaks |

**at date when at least half of tray is of harvestable maturity in break

### Objective Description of the Variety ‘RO12756’

The following detailed description sets forth the breeding procedures and the characteristics of new cultivar designated ‘RO12756’. The strain is maintained and propagated as vegetative mycelium. Spawn (inoculum for the substrate compost) is prepared from the vegetative mycelium. The new variety was tested on a scale of hundreds of individual mushrooms. The variety has shown uniformity and stability for the traits, within the limits of expected environmental influence for the traits.

All homokaryons utilized are strains of Agaricus bisporus.

In a first cross, a bridging cross strain 4x29 slow grower (sg) was crossed with a commercial off-white hybrid strain protoplast.

‘RO12756’ was produced through the cross of a homokaryon SSI of the first cross to a homokaryon SSI of strain ‘RO10425’.

‘RO12756’ sets pins and produces harvestable mushrooms as fast or faster than available brown commercial strains. ‘RO12756’ also exhibits high tray coverage, high yield, a desirably light brown cap color, medium firmness, and thick stipes and cap flesh relative to cap diameter. ‘RO12756’ additionally exhibits rounder cap shape, thicker cap apex flesh, thicker cap edge flesh, and Percent Harvested Speed faster or as fast as other available brown commercial strains.

Other characteristics of ‘RO12756’ distinguishable from other varieties are described below (compared to commercial line ‘BR06’ and commercial line ‘B14528’).

### Comparisons to Commercial Mushroom Varieties

### Initial Trials

### Tray-Level Comparisons

In further evidence of the distinctness and superiority of mushroom strain ‘RO12756’, provided below are the results of trials conducted across multiple strains' yielded trays of mushrooms. These trials compare timing for trays to reach pinset and harvestable maturity, tray coverage, cap color darkness, cap firmness, cap roundness (visual), yield at each break, and percentage of each break's yield harvested by 17 days post-casing (1st break), 24 days post-casing (2nd break), and 32 days post-casing (3rd break). Characteristics were measured from 74-147 trays of strain ‘RO12756’ mushrooms with corresponding measurements taken of 94-166 trays of strain ‘BR06’ mushrooms (Table 2A and 2B) and 12-28 trays of strain ‘B14528’ mushrooms (Table 3A and 3B). Percent Harvest Speed characteristics were measured for 34-36 trials containing strain ‘RO12756’, 33-36 trials containing strain ‘BR06’, and 3-4 trials containing strain ‘B14528’.

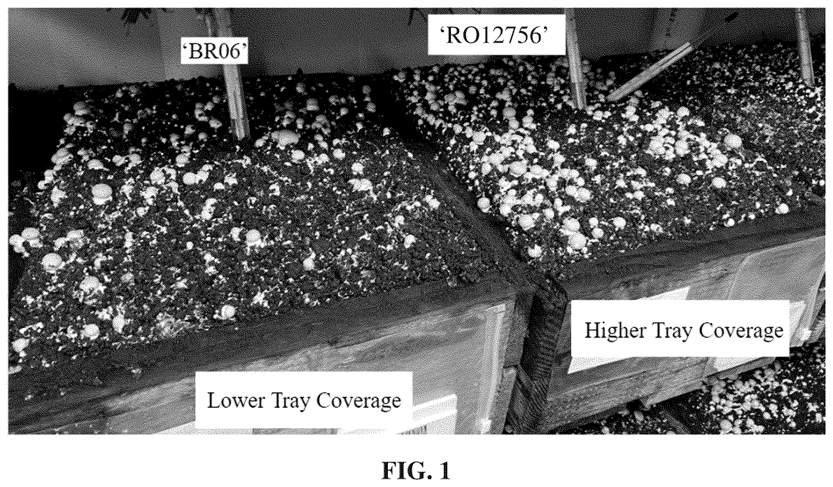

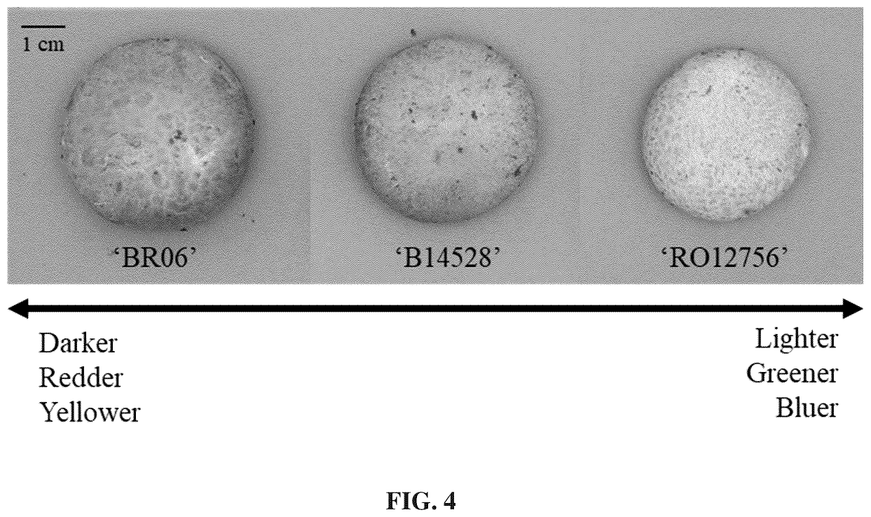

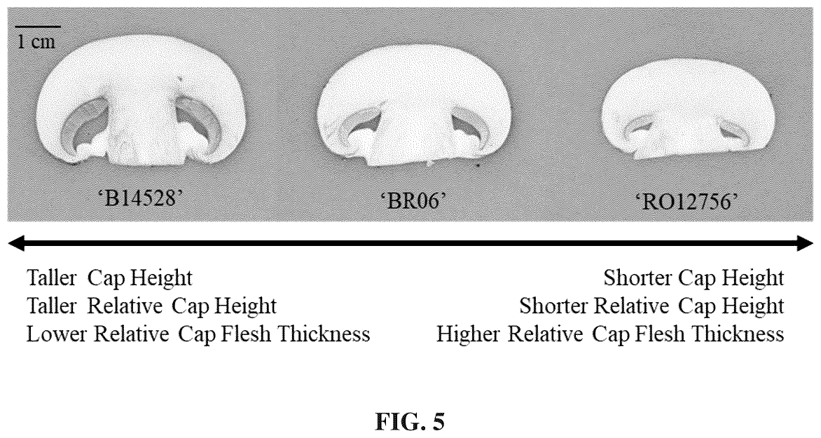

In addition to the comparisons made below, FIGS. 1 and FIGS. 4-5 visually compare variety ‘RO12756’ to other varieties in tray coverage (FIG. 1), cap color (FIG. 4), and qualities regarding cap height and cap flesh thickness (FIG. 5).

Table 2A below provides qualitative comparisons between mushroom strain ‘RO12756’ and strain ‘BR06’. Column 1 lists the tray-level or trial-level characteristic (as described in Table 1), column 2 shows the qualitative descriptor for the characteristic for mushroom strain ‘RO12756’ relative to ‘BR06’, and column 3 shows the qualitative descriptor for mushroom strain ‘BR06’ relative to ‘RO12756’.

TABLE 2A

| | | |
|---|---|---|
| Characteristic | ‘RO12756’ | ‘BR06’ |

Before First Break

| | | |
|---|---|---|
| Days to Pinset | Faster | Slower |

First Break

| | | |
|---|---|---|
| Days to 1st Break | Faster | Slower |
| Tray Coverage | Higher | Lower |
| Cap Color Darkness | Lighter | Darker |
| Cap Firmness | Softer | Firmer |
| Cap Roundness (Visual) | Flatter | Rounder |
| Percent Harvested Speed | Similar | Similar |
| Yield (lb/ft²) | Similar | Similar |

Second Break

| | | |
|---|---|---|
| Days to 2nd Break | Faster | Slower |
| Tray Coverage | Similar | Similar |
| Cap Color Darkness | Lighter | Darker |
| Cap Firmness | Softer | Firmer |
| Cap Roundness (Visual) | Flatter | Rounder |
| Percent Harvested Speed | Higher | Lower |
| Yield (lb/ft²) | Similar | Similar |

Third Break

| | | |
|---|---|---|
| Percent Harvested Speed | Higher | Lower |
| Yield (lb/ft²) | Higher | Lower |

All Breaks Combined

| | | |
|---|---|---|
| Yield (lb/ft²) | Higher | Lower |

Table 2B below provides initial quantitative comparisons between mushroom strain ‘RO12756’ and strain ‘BR06’. Column 1 lists the tray-level or trial-level characteristics (as described in Table 1), column 2 shows the average value of the characteristic for mushroom strain ‘RO12756’, column 3 shows the standard deviation of the characteristic for mushroom strain ‘RO12756’, column 4 shows the average value of the characteristic for mushroom strain ‘BR06’, column 5 shows the standard deviation of the characteristic for mushroom strain ‘BR06’, and column 6 shows the p-values of two-sample, two-sided t-tests. Bolded p-values indicate p<0.05.

TABLE 2B

| | ‘RO12756’ | | ‘BR06’ | | |
|---|---|---|---|---|---|
| Characteristic | Average | St. Dev. | Average | St. Dev. | p-value |

Before First Break

| | | | | | |
|---|---|---|---|---|---|
| Days to Pinset | 11.722 | 0.999 | 12.316 | 0.880 | 4.139E−05 |

First Break

| | | | | | |
|---|---|---|---|---|---|
| Days to 1st Break | 17.317 | 0.752 | 17.598 | 0.735 | 1.153E−02 |
| Tray Coverage | 4.178 | 0.799 | 3.935 | 0.866 | 2.900E−02 |
| Cap Color Darkness | 2.916 | 0.601 | 3.872 | 0.380 | 6.840E−35 |
| Cap Firmness | 3.543 | 0.519 | 4.154 | 0.425 | 4.790E−19 |
| Cap Roundness (Visual) | 2.848 | 0.387 | 2.984 | 0.127 | 2.930E−04 |
| Percent Harvested Speed | 24.105% | 23.663 | 16.868% | 19.090 | 1.241E−01 |
| Yield (lb/ft²) | 2.483 | 0.701 | 2.523 | 1.017 | 6.860E−01 |

Second Break

| | | | | | |
|---|---|---|---|---|---|
| Days to 2nd Break | 24.824 | 0.800 | 25.274 | 0.675 | 1.122E−04 |
| Tray Coverage | 4.000 | 0.611 | 4.150 | 0.605 | 6.140E−02 |
| Cap Color Darkness | 3.009 | 0.427 | 3.859 | 0.349 | 9.160E−45 |
| Cap Firmness | 3.509 | 0.553 | 3.969 | 0.435 | 9.200E−12 |
| Cap Roundness (Visual) | 2.634 | 0.484 | 2.992 | 0.154 | 1.030E−13 |
| Percent Harvested Speed | 42.447% | 25.152 | 25.151% | 23.811 | 1.431E−03 |
| Yield (lb/ft²) | 1.606 | 0.537 | 1.601 | 0.617 | 9.340E−01 |

Third Break

| | | | | | |
|---|---|---|---|---|---|
| Percent Harvested Speed | 63.200% | 26.702 | 34.306% | 32.035 | 2.320E−05 |
| Yield (lb/ft²) | 1.038 | 0.313 | 0.747 | 0.371 | 9.180E−13 |

All Breaks Combined

| | | | | | |
|---|---|---|---|---|---|
| Yield (lb/ft²) | 5.127 | 0.792 | 4.871 | 0.918 | 9.080E−03 |

Table 3A below provides qualitative comparisons between mushroom strain ‘RO12756’ and strain ‘B14528’. Column 1 lists the tray-level or trial-level characteristic (as described in Table 1), column 2 shows the qualitative descriptor for the characteristic for mushroom strain ‘RO12756’ relative to ‘B14528’, and column 3 shows the qualitative descriptor for mushroom strain ‘B14528’ relative to ‘RO12756’.

TABLE 3A

| | | |
|---|---|---|
| Characteristic | ‘RO12756’ | ‘B14528’ |

Before First Break

| | | |
|---|---|---|
| Compost Colonization | Higher | Lower |
| First Break | | |
| Days to 1st Break | Similar | Similar |
| Tray Coverage | Similar | Similar |
| Cap Color Darkness | Lighter | Darker |
| Cap Firmness | Softer | Firmer |
| Cap Roundness (Visual) | Similar | Similar |
| Percent Harvested Speed | Similar | Similar |

Second Break

| | | |
|---|---|---|
| Days to 2nd Break | Similar | Similar |
| Tray Coverage | Higher | Lower |
| Cap Color Darkness | Lighter | Darker |
| Cap Firmness | Softer | Firmer |
| Cap Roundness (Visual) | Similar | Similar |
| Percent Harvested Speed | Similar | Similar |

Table 3B below provides initial quantitative comparisons between mushroom strain ‘RO12756’ and strain ‘B14528’. Column 1 lists the tray-level or trial-level characteristics (as described in Table 1), column 2 shows the average value of the characteristic for mushroom strain ‘RO12756’, column 3 shows the standard deviation of the characteristic for mushroom strain ‘RO12756’, column 4 shows the average value of the characteristic for mushroom strain ‘B14528’, column 5 shows the standard deviation of the characteristic for mushroom strain ‘B14528’, and column 6 shows the p-values of two-sample, two-sided t-tests. Bolded p-values indicate p<0.05.

TABLE 3B

| | ‘RO12756’ | | ‘B14528’ | | |
|---|---|---|---|---|---|
| Characteristic | Average | St. Dev. | Average | St. Dev. | p-value |

Before First Break

| | | | | | |
|---|---|---|---|---|---|
| Compost Colonization | 4.350 | 0.737 | 3.643 | 1.026 | 6.920E−05 |
| First Break | | | | | |
| Days to 1st Break | 17.317 | 0.752 | 17.000 | 0.649 | 8.599E−02 |
| Tray Coverage | 4.178 | 0.799 | 4.000 | 0.471 | 2.630E−01 |
| Cap Color Darkness | 2.916 | 0.601 | 4.000 | 0.272 | 4.050E−16 |
| Cap Firmness | 3.543 | 0.519 | 3.786 | 0.418 | 2.400E−02 |
| Cap Roundness (Visual) | 2.848 | 0.387 | 2.929 | 0.262 | 2.990E−01 |
| Percent Harvested Speed | 24.105% | 23.660 | 19.000% | 11.247 | 4.392E−01 |

Second Break

| | | | | | |
|---|---|---|---|---|---|
| Days to 2nd Break | 24.824 | 0.800 | 24.417 | 0.669 | 9.848E−02 |
| Tray Coverage | 4.000 | 0.611 | 3.250 | 0.550 | 1.150E−06 |
| Cap Color Darkness | 3.009 | 0.427 | 3.929 | 0.378 | 2.670E−19 |
| Cap Firmness | 3.509 | 0.553 | 3.857 | 0.448 | 2.470E−03 |
| Cap Roundness (Visual) | 2.634 | 0.484 | 2.536 | 0.508 | 3.430E−01 |
| Percent Harvested Speed | 42.447% | 25.152 | 39.000% | 34.438 | 8.380E−01 |

### Individual Mushroom-Level Comparisons

In additional evidence of the distinctness and superiority of mushroom strain ‘RO12756’, provided below is a summary of precise quantitative measurements of individual mushrooms at first and second breaks. These data compare Cap L* Value, Cap a* Value, and Cap b* Values in the International Commission on Illumination (CIELAB) color space, Cap Diameter, Cap Height, Cap Flesh Thickness, Stipe Diameter, Relative Cap Height, Relative Cap Flesh Thickness, and Relative Stipe Diameter (as described in Table 1). Measurements from 390 first break and 100 second break ‘RO12756’ mushrooms were compared to 400 first break and 120 second break ‘BR06’ mushrooms (Tables 14A and 14B) and 150 first break and 80 second break ‘B14528’ mushrooms (Tables 15A and 15B).

Table 4A below provides qualitative comparisons between mushroom strain ‘RO12756’ and strain ‘BR06’. Column 1 lists the mushroom-level characteristics (as described in Table 1), column 2 shows the qualitative descriptor for the characteristic for mushroom strain ‘RO12756’ relative to ‘BR06’, and column 3 shows the qualitative descriptor for mushroom strain ‘BR06’ relative to ‘RO12756’.

TABLE 4A

| | | |
|---|---|---|
| Characteristic | ‘RO12756’ | ‘BR06’ |

First Break

| | | |
|---|---|---|
| Cap L* Value (dark-light) | Lighter | Darker |
| Cap a* Value (green-red) | Greener | Redder |
| Cap b* Value (blue-yellow) | Bluer | Yellower |
| Cap Diameter | Smaller | Larger |
| Cap Height | Shorter | Taller |
| Cap Flesh Thickness | Similar | Similar |
| Stipe Diameter | Smaller | Larger |
| Relative Cap Height | Shorter | Taller |
| Relative Cap Flesh Thickness | Thicker | Thinner |
| Relative Stipe Diameter | Smaller | Larger |

Second Break

| | | |
|---|---|---|
| Cap L* Value (dark-light) | Lighter | Darker |
| Cap a* Value (green-red) | Greener | Redder |
| Cap b* Value (blue-yellow) | Bluer | Yellower |
| Cap Diameter | Similar | Similar |
| Cap Height | Shorter | Taller |
| Cap Flesh Thickness | Similar | Similar |
| Stipe Diameter | Similar | Similar |
| Relative Cap Height | Shorter | Taller |
| Relative Cap Flesh Thickness | Similar | Similar |
| Relative Stipe Diameter | Similar | Similar |

Table 4B below provides initial quantitative comparisons between mushroom strain ‘RO12756’ and strain ‘BR06’. Column 1 lists the mushroom-level characteristics (as described in Table 1), column 2 shows the average value of the characteristic for mushroom strain ‘RO12756’, column 3 shows the standard deviation of the characteristic for mushroom strain ‘RO12756’, column 4 shows the average value of the characteristic for mushroom strain ‘BR06’, column 5 shows the standard deviation of the characteristic for mushroom strain ‘BR06’, and column 6 shows the p-values of two-sample, two-sided t-tests. Bolded p-values indicate p<0.05.

TABLE 4B

| | ‘RO12756’ | | ‘BR06’ | | |
|---|---|---|---|---|---|
| Characteristic | Average | St. Dev. | Average | St. Dev. | p-value |

First Break

| | | | | | |
|---|---|---|---|---|---|
| Cap L* Value (dark-light) | 74.237 | 3.524 | 64.335 | 5.707 | 5.943E−128 |
| Cap a* Value (green-red) | 3.899 | 1.581 | 7.605 | 1.770 | 1.242E−138 |
| Cap b* Value (blue-yellow) | 24.770 | 2.559 | 28.453 | 2.451 | 3.905E−76 |
| Cap Diameter | 40.571 | 3.829 | 42.200 | 4.005 | 7.566E−09 |
| Cap Height | 24.199 | 1.994 | 25.455 | 2.059 | 1.797E−17 |
| Cap Flesh Thickness | 13.069 | 1.395 | 13.130 | 1.313 | 5.270E−01 |
| Stipe Diameter | 18.258 | 2.139 | 19.546 | 2.585 | 1.130E−13 |
| Relative Cap Height | 0.598 | 0.033 | 0.605 | 0.031 | 3.410E−03 |
| Relative Cap Flesh Thickness | 0.323 | 0.028 | 0.312 | 0.026 | 3.060E−08 |
| Relative Stipe Diameter | 0.451 | 0.040 | 0.464 | 0.054 | 9.166E−05 |

Second Break

| | | | | | |
|---|---|---|---|---|---|
| Cap L* Value (dark-light) | 73.318 | 3.497 | 66.428 | 4.274 | 1.03E−28 |
| Cap a* Value (green-red) | 4.282 | 1.216 | 6.359 | 1.461 | 1.17E−23 |
| Cap b* Value (blue-yellow) | 24.796 | 3.168 | 27.600 | 2.440 | 2.72E−12 |
| Cap Diameter | 42.833 | 5.946 | 43.005 | 4.063 | 8.00E−01 |
| Cap Height | 24.156 | 2.112 | 25.313 | 2.234 | 1.19E−04 |
| Cap Flesh Thickness | 12.359 | 1.048 | 12.526 | 1.163 | 2.71E−01 |
| Stipe Diameter | 16.115 | 1.904 | 15.878 | 2.022 | 3.75E−01 |
| Relative Cap Height | 0.569 | 0.053 | 0.590 | 0.035 | 6.90E−04 |
| Relative Cap Flesh Thickness | 0.293 | 0.040 | 0.292 | 0.025 | 9.06E−01 |
| Relative Stipe Diameter | 0.379 | 0.038 | 0.370 | 0.035 | 5.72E−02 |

Table 5A below provides qualitative comparisons between mushroom strain ‘RO12756’ and strain ‘B14528’. Column 1 lists the mushroom-level characteristics (as described in Table 1), column 2 shows the qualitative descriptor for the characteristic for mushroom strain ‘RO12756’ relative to ‘B14528’, and column 3 shows the qualitative descriptor for mushroom strain ‘B14528’ relative to ‘RO12756’.

TABLE 5A

| | | |
|---|---|---|
| Characteristic | ‘RO12756’ | ‘B14528’ |

First Break

| | | |
|---|---|---|
| Cap L* Value (dark-light) | Lighter | Darker |
| Cap a* Value (green-red) | Greener | Redder |
| Cap b* Value (blue-yellow) | Bluer | Yellower |
| Cap Diameter | Smaller | Larger |
| Cap Height | Shorter | Taller |
| Cap Flesh Thickness | Similar | Similar |
| Stipe Diameter | Larger | Smaller |
| Relative Cap Height | Shorter | Taller |
| Relative Cap Flesh Thickness | Thicker | Thinner |
| Relative Stipe Diameter | Larger | Smaller |

Second Break

| | | |
|---|---|---|
| Cap L* Value (dark-light) | Lighter | Darker |
| Cap a* Value (green-red) | Greener | Redder |
| Cap b* Value (blue-yellow) | Bluer | Yellower |
| Cap Diameter | Similar | Similar |
| Cap Height | Shorter | Taller |
| Cap Flesh Thickness | Thicker | Thinner |
| Stipe Diameter | Larger | Smaller |
| Relative Cap Height | Shorter | Taller |
| Relative Cap Flesh Thickness | Similar | Similar |
| Relative Stipe Diameter | Larger | Smaller |

Table 5B below provides quantitative comparisons between mushroom strain ‘RO12756’ and strain ‘B14528’. Column 1 lists the mushroom-level characteristics (as described in Table 1), column 2 shows the average value of the characteristic for mushroom strain ‘RO12756’, column 3 shows the standard deviation of the characteristic for mushroom strain ‘RO12756’, column 4 shows the average value of the characteristic for mushroom strain ‘B14528’, column 5 shows the standard deviation of the characteristic for mushroom strain ‘B14528’, and column 6 shows the p-values of two-sample, two-sided t-tests. Bolded p-values indicate p<0.05.

TABLE 5B

| | ‘RO12756’ | | ‘B14528’ | | |
|---|---|---|---|---|---|
| Characteristic | Average | St. Dev. | Average | St. Dev. | p-value |

First Break

| | | | | | |
|---|---|---|---|---|---|
| Cap L* Value (dark-light) | 74.237 | 3.524 | 67.579 | 5.925 | 1.560E−47 |
| Cap a* Value (green-red) | 3.899 | 1.581 | 5.987 | 1.854 | 3.850E−34 |
| Cap b* Value (blue-yellow) | 24.770 | 2.559 | 27.637 | 2.363 | 3.510E−29 |
| Cap Diameter | 40.571 | 3.829 | 43.218 | 3.827 | 6.590E−13 |
| Cap Height | 24.199 | 1.994 | 26.568 | 1.872 | 2.430E−33 |
| Cap Flesh Thickness | 13.069 | 1.395 | 13.057 | 1.012 | 9.210E−01 |
| Stipe Diameter | 18.258 | 2.139 | 17.787 | 2.018 | 1.790E−02 |
| Relative Cap Height | 0.598 | 0.033 | 0.616 | 0.030 | 2.260E−09 |
| Relative Cap Flesh Thickness | 0.323 | 0.028 | 0.303 | 0.024 | 3.050E−14 |
| Relative Stipe Diameter | 0.451 | 0.040 | 0.413 | 0.042 | 1.900E−21 |

Second Break

| | | | | | |
|---|---|---|---|---|---|
| Cap L* Value (dark-light) | 73.318 | 3.497 | 68.288 | 5.217 | 8.20E−13 |
| Cap a* Value (green-red) | 4.282 | 1.216 | 5.256 | 1.241 | 3.51E−07 |
| Cap b* Value (blue-yellow) | 24.796 | 3.168 | 26.285 | 2.185 | 4.48E−04 |
| Cap Diameter | 42.833 | 5.946 | 41.712 | 3.417 | 1.35E−01 |
| Cap Height | 24.156 | 2.112 | 25.062 | 2.039 | 4.14E−03 |
| Cap Flesh Thickness | 12.359 | 1.048 | 11.868 | 1.370 | 7.02E−03 |
| Stipe Diameter | 16.115 | 1.904 | 14.725 | 1.442 | 2.04E−07 |
| Relative Cap Height | 0.569 | 0.053 | 0.602 | 0.033 | 4.09E−06 |
| Relative Cap Flesh Thickness | 0.293 | 0.040 | 0.286 | 0.036 | 2.14E−01 |
| Relative Stipe Diameter | 0.379 | 0.038 | 0.355 | 0.044 | 1.48E−04 |

### Additional Trials

### Tray-Level Comparisons

Table 6A below provides qualitative comparisons between mushroom strain ‘RO12756’ and strain ‘BR06’. Traits of particular interest are bolded. Column 1 lists the tray-level or trial-level characteristic (as described in Table 1), column 2 shows the qualitative descriptor for the characteristic for mushroom strain ‘RO12756’ relative to ‘BR06’, and column 3 shows the qualitative descriptor for mushroom strain ‘BR06’ relative to ‘RO12756’.

TABLE 6A

| | | |
|---|---|---|
| Characteristic | ‘RO12756’ | ‘BR06’ |

Before First Break

| | | |
|---|---|---|
| Days to Pinset | Faster | Slower |

First Break

| | | |
|---|---|---|
| Days to 1st Break | Faster | Slower |
| Percent Harvested Speed | Faster | Slower |
| [symbol] [symbol] | Similar | Similar |

Second Break

| | | |
|---|---|---|
| Days to 2nd Break | Faster | Slower |
| [symbol] [symbol] | Higher | Lower |

Third Break

| | | |
|---|---|---|
| [symbol] [symbol] | Higher | Lower |

All Breaks Combined

| | | |
|---|---|---|
| [symbol] [symbol] | Higher | Lower |

Table 6B below provides quantitative comparisons between mushroom strain ‘RO12756’ and strain ‘BR06’.

The first part of analyzing trait measurements in the additional trials herein included making comparisons between all three of instant strain ‘RO12756’, comparative strain ‘BR06’, and comparative strain ‘B14528’. For these three strains, a Shapiro-Wilk test and a Levene test were performed first to assess if the data satisfy the assumptions of normality and equal variances among groups, respectively, of a standard ANOVA. The Shapiro-Wilk test and Levene test determined that most of these traits satisfied neither assumption. Therefore, a Kruskal-Wallis test (Kruskal, W. H., & Wallis, W. A. (1952). Use of ranks in one-criterion variance analysis. Journal of the American statistical Association, 47(260), 583-621), a non-parametric variation of ANOVA that does not assume normality, equal variances among groups, or equal sample sizes among groups, was performed. Medians and interquartile ranges (IQR) were reported, as these metrics are robust to non-normal distributions. Means are also reported for consistency with normally-distributed traits.

The Shapiro-Wilk test and Levene test determined that most developmental timing traits (Days to Pinset, Days to 1st Break, and Days to 2nd Break) and yield traits were not normally distributed and variances among groups were not equal. The Kruskal-Wallis test and Dunn post-hoc test (“Dunn's Test”; Dunn, O. J. (1964). Multiple comparisons using rank sums. Technometrics, 6(3), 241-252) were used in these cases. When the Kruskal-Wallis test results were significant based on a threshold of p<0.05, Dunn's test was performed post-hoc to make pairwise strain comparisons (Dunn, 1964). P-values were adjusted using the Bonferroni correction to minimize risk of Type 1 error (false significance). An exception to this trend was Percent Harvested Speed, which was normally distributed and exhibited equal variance for all strains. For assessing Percent Harvested Speed, a standard ANOVA was used and a Tukey Honestly Significant Difference (HSD) test was performed post-hoc for pairwise strain comparisons. Standard deviations multiplied by 100 for consistency alongside percentages (“SD*100”) were reported for this trait. This testing was performed on groups of three strains, but the results show a pairwise subset of the tests for consistency with the initial trials.

In Table 6B, column 1 shows each trait and the strains compared for said trait, column 2 shows the mean value for that strain's measurements of that trait, column 3 shows the median value of the same measurements, column 4 shows the interquartile range (“IQR”) for the same measurements, column 5 shows the number of trials conducted, and column 6 shows the statistical significance group calculated for that strain for that trait according to Dunn's Test. Traits of particular interest are bolded.

TABLE 6B

| | | | | | |
|---|---|---|---|---|---|
| | | | | | Significance |
| | | | | Number of | Group |
| Variety | Mean | Median | IQR | Trials | (Dunn's Test) |

Before First Break
Days to Pinset

| | | | | | |
|---|---|---|---|---|---|
| ‘RO12756’ | 11.4 | 11.0 | 1.00 | 8 | a |
| ‘BR06’ | 12.6 | 13.0 | 1.00 | 8 | b |

First Break
Days to 1st Break

| | | | | | |
|---|---|---|---|---|---|
| ‘RO12756’ | 17.5 | 17.0 | 1.00 | 8 | a |
| ‘BR06’ | 18.5 | 18.0 | 1.00 | 8 | b |

[symbol]

[symbol]

| | | | | | |
|---|---|---|---|---|---|
| ‘RO12756’ | 11.91 | 12.40 | 4.88 | 8 | a |
| ‘BR06’ | 11.96 | 12.69 | 9.52 | 8 | a |

Second Break
Days to 2nd Break

| | | | | | |
|---|---|---|---|---|---|
| ‘RO12756’ | 24.8 | 25.0 | 2.00 | 7 | a |
| ‘BR06’ | 25.5 | 25.0 | 1.00 | 7 | b |

[symbol]

[symbol]

| | | | | | |
|---|---|---|---|---|---|
| ‘RO12756’ | 8.54 | 8.15 | 2.64 | 8 | a |
| ‘BR06’ | 7.37 | 5.96 | 4.39 | 8 | b |

Third Break

[symbol]

[symbol]

| | | | | | |
|---|---|---|---|---|---|
| ‘RO12756’ | 4.78 | 4.83 | 1.86 | 8 | a |
| ‘BR06’ | 3.66 | 2.78 | 2.78 | 8 | b |

All Breaks Combined

[symbol]

[symbol]

| ‘RO12756’ | 25.29 | 24.95 | 4.15 | 8 | a |
|---|---|---|---|---|---|
| ‘BR06’ | 23.00 | 23.48 | 5.81 | 8 | b |
| | | | | | Significance |
| Percent Harvested Speed | | | | | Group |
| (measured i nFirst Break) | | | SD * 100 | | (Tukey HSD) |
| ‘RO12756’ | 48.2% | 50.9% | 16.30 | 8 | b |
| ‘BR06’ | 28.6% | 27.4% | 20.90 | 8 | a |

Table 7A below provides qualitative comparisons between mushroom strain ‘RO12756’ and strain ‘B14528’. Traits of particular interest are bolded. Column 1 lists the tray-level or trial-level characteristic (as described in Table 1), column 2 shows the qualitative descriptor for the characteristic for mushroom strain ‘RO12756’ relative to ‘B14528’, and column 3 shows the qualitative descriptor for mushroom strain ‘B14528’ relative to ‘RO12756’.

TABLE 7A

| | | |
|---|---|---|
| Characteristic | ‘RO12756’ | ‘B14528’ |

Before First Break

| | | |
|---|---|---|
| Days to Pinset | Similar | Similar |

First Break

| | | |
|---|---|---|
| Days to 1st Break | Similar | Similar |
| Percent Harvested Speed | Similar | Similar |
| [symbol] [symbol] | Similar | Similar |

Second Break

| | | |
|---|---|---|
| Days to 2nd Break | Similar | Similar |
| [symbol] [symbol] | Similar | Similar |

Third Break

| | | |
|---|---|---|
| [symbol] [symbol] | Similar | Similar |

All Breaks Combined

| | | |
|---|---|---|
| [symbol] [symbol] | Similar | Similar |

Table 7B below provides additional quantitative comparison between mushroom strain ‘RO12756’ and strain ‘B14528’. Column 1 shows the strains compared for each trait, column 2 shows the mean value for that strain's measurements, column 3 shows the median value of the same measurements, column 4 shows either the interquartile range (“IQR”) or the standard deviation multiplied by 100 for consistency alongside percentages (“SD*100”) for the same measurements, column 5 shows the number of trials conducted, and column 6 shows the statistical significance group calculated for that strain for that trait according to either Dunn's Test or Tukey HSD.

TABLE 7B

| | | | | | |
|---|---|---|---|---|---|
| | | | | Number | Significance |
| | | | | of | Group |
| Characteristic | Mean | Median | IQR | Trials | (Dunn's Test) |

Before First Break
Days to Pinset

| | | | | | |
|---|---|---|---|---|---|
| ‘RO12756’ | 11.40 | 11.00 | 1.00 | 8 | a |
| ‘B14528’ | 11.40 | 11.00 | 1.00 | 8 | a |

First Break
Days to 1st Break

| | | | | | |
|---|---|---|---|---|---|
| ‘RO12756’ | 17.50 | 17.00 | 1.00 | 8 | a |
| ‘B14528’ | 17.50 | 17.00 | 1.00 | 8 | a |

[symbol]

[symbol]

| | | | | | |
|---|---|---|---|---|---|
| ‘RO12756’ | 11.91 | 12.40 | 4.88 | 8 | a |
| ‘B14528’ | 12.55 | 12.25 | 5.27 | 8 | a |

Second Break
Days to 2nd Break

| | | | | | |
|---|---|---|---|---|---|
| ‘RO12756’ | 24.80 | 25.00 | 2.00 | 7 | a |
| ‘B14528’ | 25.00 | 25.00 | 0.50 | 7 | a |

[symbol]

[symbol]

| | | | | | |
|---|---|---|---|---|---|
| ‘RO12756’ | 8.54 | 8.15 | 2.64 | 8 | a |
| ‘B14528’ | 8.45 | 8.01 | 2.39 | 8 | a |

Third Break

[symbol]

[symbol]

| | | | | | |
|---|---|---|---|---|---|
| ‘RO12756’ | 4.78 | 4.83 | 1.86 | 8 | a |
| ‘B14528’ | 5.52 | 5.57 | 2.88 | 8 | a |

All Breaks Combined

[symbol]

[symbol]

| ‘RO12756’ | 25.29 | 24.95 | 4.15 | 8 | a |
|---|---|---|---|---|---|
| ‘B14528’ | 26.51 | 27.10 | 5.76 | 8 | a |
| Percent | | | | | |
| Harvested Speed | | | | | Significance |
| (measured in | | | | | Group |
| First Break) | Mean | Median | SD * 100 | | (Tukey HSD) |
| ‘RO12756’ | 48.2% | 50.9% | 16.30 | 8 | b |
| ‘B14528’ | 53.5% | 55.7% | 14.00 | 8 | b |

### Individual Mushroom-Level Comparisons

In additional evidence of the distinctness and superiority of mushroom strain ‘RO12756’, provided below is a summary of precise quantitative measurements of individual mushrooms at first and second breaks. These data compare Cap L* Value, Cap a* Value, and Cap b* Values in the International Commission on Illumination (CIELAB) color space, Cap Apex Flesh Thickness, Cap Edge Flesh Thickness, and Cap Roundness (Calculated) (as described in Table 1). Measurements were taken for hundreds of mushrooms per trait, noted as sample size (n) in Tables 8B and 9B.

Table 8A below provides qualitative comparisons between mushroom strain ‘RO12756’ and strain ‘BR06’. Traits of particular interest are bolded. Column 1 lists the mushroom-level characteristics (as described in Table 1), column 2 shows the qualitative descriptor for the characteristic for mushroom strain ‘RO12756’ relative to ‘BR06’, and column 3 shows the qualitative descriptor for mushroom strain ‘BR06’ relative to ‘RO12756’.

TABLE 8A

| | | |
|---|---|---|
| Characteristic | ‘RO12756’ | ‘BR06’ |

First Break

| | | |
|---|---|---|
| Cap L* Value (dark-light) | Lighter | Darker |
| Cap a* Value (green-red) | Greener | Redder |
| Cap b* Value (blue-yellow) | Bluer | Yellower |
| Cap Apex Flesh Thickness | Thicker | Thinner |
| Cap Edge Flesh Thickness | Similar | Similar |
| Cap Roundness (Calculated) | Similar | Similar |

Second Break

| | | |
|---|---|---|
| Cap L* Value (dark-light) | Lighter | Darker |
| Cap a* Value (green-red) | Greener | Redder |
| Cap b* Value (blue-yellow) | Bluer | Yellower |
| Cap Apex Flesh Thickness | Thicker | Thinner |
| Cap Edge Flesh Thickness | Thicker | Thinner |
| Cap Roundness (Calculated) | Flatter | Rounder |

Table 8B below provides additional quantitative comparisons between mushroom strain ‘RO12756’ and strain ‘BR06’. Column 1 shows each trait and the strains compared for said trait, column 2 shows the mean value for that strain's measurements of that trait, column 3 shows the median value of the same measurements, column 4 shows the interquartile range (“IQR”) for the same measurements, column 5 shows the number of mushrooms assessed (n), and column 6 shows the statistical significance group calculated for that strain for that trait according to Dunn's Test.

TABLE 8B

| | | | | | |
|---|---|---|---|---|---|
| | | | | Number of | Significance |
| | | | | Mushrooms | Group |
| Variety | Mean | Median | IQR | (n) | (Dunn's Test) |

First Break
Cap L* Value (dark-light)

| | | | | | |
|---|---|---|---|---|---|
| ‘RO12756’ | 72.12 | 72.29 | 4.81 | 580 | a |
| ‘BR06’ | 63.30 | 62.74 | 9.83 | 797 | b |

Cap a* Value (green-red)

| | | | | | |
|---|---|---|---|---|---|
| ‘RO12756’ | 5.62 | 5.66 | 1.88 | 580 | a |
| ‘BR06’ | 8.67 | 8.96 | 2.58 | 787 | b |

Cap b* Value (blue-yellow)

| | | | | | |
|---|---|---|---|---|---|
| ‘RO12756’ | 26.98 | 27.06 | 4.11 | 580 | a |
| ‘BR06’ | 28.72 | 29.04 | 3.17 | 776 | b |

Cap Apex Flesh Thickness

| | | | | | |
|---|---|---|---|---|---|
| ‘RO12756’ | 0.55 | 0.55 | 0.06 | 575 | a |
| ‘BR06’ | 0.52 | 0.52 | 0.05 | 541 | b |

Cap Edge Flesh Thickness

| | | | | | |
|---|---|---|---|---|---|
| ‘RO12756’ | 0.22 | 0.23 | 0.10 | 569 | a |
| ‘BR06’ | 0.21 | 0.21 | 0.09 | 537 | a |

Cap Roundness (Calculated)

| | | | | | |
|---|---|---|---|---|---|
| ‘RO12756’ | 0.61 | 0.61 | 0.04 | 579 | a |
| ‘BR06’ | 0.61 | 0.61 | 0.04 | 541 | a |

Second Break
Cap L* Value (dark-light)

| | | | | | |
|---|---|---|---|---|---|
| ‘RO12756’ | 73.02 | 72.87 | 4.35 | 200 | a |
| ‘BR06’ | 65.68 | 65.49 | 5.90 | 340 | b |

Cap a* Value (green-red)

| | | | | | |
|---|---|---|---|---|---|
| ‘RO12756’ | 4.84 | 4.95 | 1.64 | 200 | a |
| ‘BR06’ | 7.21 | 7.31 | 2.42 | 340 | b |

Cap b* Value (blue-yellow)

| | | | | | |
|---|---|---|---|---|---|
| ‘RO12756’ | 26.55 | 26.83 | 3.24 | 200 | a |
| ‘BR06’ | 28.15 | 28.49 | 2.86 | 340 | b |

Cap Apex Flesh Thickness

| | | | | | |
|---|---|---|---|---|---|
| ‘RO12756’ | 0.54 | 0.54 | 0.04 | 198 | a |
| ‘BR06’ | 0.51 | 0.52 | 0.07 | 362 | b |

Cap Edge Flesh Thickness

| | | | | | |
|---|---|---|---|---|---|
| ‘RO12756’ | 0.27 | 0.27 | 0.10 | 199 | a |
| ‘BR06’ | 0.23 | 0.23 | 0.09 | 362 | b |

Cap Roundness (Calculated)

| | | | | | |
|---|---|---|---|---|---|
| ‘RO12756’ | 0.56 | 0.56 | 0.05 | 199 | a |
| ‘BR06’ | 0.60 | 0.59 | 0.06 | 363 | b |

Table 9A below provides qualitative comparisons between mushroom strain ‘RO12756’ and strain ‘B14528’. Column 1 lists the mushroom-level characteristics (as described in Table 1), column 2 shows the qualitative descriptor for the characteristic for mushroom strain ‘RO12756’ relative to ‘B14528’, and column 3 shows the qualitative descriptor for mushroom strain ‘B14528’ relative to ‘RO12756’.

TABLE 9A

| | | |
|---|---|---|
| Characteristic | ‘RO12756’ | ‘B14528’ |

First Break

| | | |
|---|---|---|
| Cap L* Value (dark-light) | Lighter | Darker |
| Cap a* Value (green-red) | Greener | Redder |
| Cap b* Value (blue-yellow) | Bluer | Yellower |
| Cap Apex Flesh Thickness | Thicker | Thinner |
| Cap Edge Flesh Thickness | Similar | Similar |
| Cap Roundness (Calculated) | Similar | Similar |

Second Break

| | | |
|---|---|---|
| Cap L* Value (dark-light) | Lighter | Darker |
| Cap a* Value (green-red) | Greener | Redder |
| Cap b* Value (blue-yellow) | Bluer | Yellower |
| Cap Apex Flesh Thickness | Thicker | Thinner |
| Cap Edge Flesh Thickness | Thicker | Thinner |
| Cap Roundness (Calculated) | Flatter | Rounder |

Table 9B shows additional quantitative comparisons between mushroom strain ‘RO12756’ and strain ‘B14528’. Column 1 lists the mushroom-level characteristics (as described in Table 1) and each strain, column 2 shows the mean value of the characteristic, column 3 shows the median value of the characteristic, column 4 shows the interquartile range (IQR) for the characteristic, column 5 shows the number of mushrooms assessed (n), and column 6 shows the statistical significance group calculated according to Dunn's Test.

TABLE 9B

| | | | | | |
|---|---|---|---|---|---|
| | | | | Number of | Significance |
| | | | | Mushrooms | Group |
| Variety | Mean | Median | IQR | (n) | (Dunn's Test) |

First Break
Cap L* Value (dark-light)

| | | | | | |
|---|---|---|---|---|---|
| ‘RO12756’ | 72.12 | 72.29 | 4.81 | 580 | a |
| ‘B14528’ | 64.06 | 64.13 | 6.56 | 340 | b |

Cap a* Value (green-red)

| | | | | | |
|---|---|---|---|---|---|
| ‘RO12756’ | 5.62 | 5.66 | 1.88 | 580 | a |
| ‘B14528’ | 8.26 | 8.31 | 2.13 | 340 | c |

Cap b* Value (blue-yellow)

| | | | | | |
|---|---|---|---|---|---|
| ‘RO12756’ | 26.98 | 27.06 | 4.11 | 580 | a |
| ‘B14528’ | 30.31 | 30.16 | 3.39 | 340 | c |

Cap Roundness (Calculated)

| | | | | | |
|---|---|---|---|---|---|
| ‘RO12756’ | 0.61 | 0.61 | 0.04 | 579 | a |
| ‘B14528’ | 0.61 | 0.61 | 0.05 | 389 | a |

Cap Apex Flesh Thickness

| | | | | | |
|---|---|---|---|---|---|
| ‘RO12756’ | 0.55 | 0.55 | 0.06 | 575 | a |
| ‘B14528’ | 0.47 | 0.46 | 0.06 | 387 | c |

Cap Edge Flesh Thickness

| | | | | | |
|---|---|---|---|---|---|
| ‘RO12756’ | 0.22 | 0.23 | 0.10 | 569 | a |
| ‘B14528’ | 0.22 | 0.22 | 0.08 | 387 | a |

Second Break
Cap L* Value (dark-light)

| | | | | | |
|---|---|---|---|---|---|
| ‘RO12756’ | 73.02 | 72.87 | 4.35 | 200 | a |
| ‘B14528’ | 68.56 | 69.02 | 6.54 | 170 | c |

Cap a* Value (green-red)

| | | | | | |
|---|---|---|---|---|---|
| ‘RO12756’ | 4.84 | 4.95 | 1.64 | 200 | a |
| ‘B14528’ | 6.09 | 5.96 | 2.05 | 170 | c |

Cap b* Value (blue-yellow)

| | | | | | |
|---|---|---|---|---|---|
| ‘RO12756’ | 26.55 | 26.83 | 3.24 | 200 | a |
| ‘B14528’ | 27.37 | 27.78 | 3.22 | 170 | c |

Cap Roundness (Calculated)

| | | | | | |
|---|---|---|---|---|---|
| ‘RO12756’ | 0.56 | 0.56 | 0.05 | 199 | a |
| ‘B14528’ | 0.60 | 0.60 | 0.05 | 198 | b |

Cap Apex Flesh Thickness

| | | | | | |
|---|---|---|---|---|---|
| ‘RO12756’ | 0.54 | 0.54 | 0.04 | 198 | a |
| ‘B14528’ | 0.46 | 0.46 | 0.05 | 198 | c |

Cap Edge Flesh Thickness

| | | | | | |
|---|---|---|---|---|---|
| ‘RO12756’ | 0.27 | 0.27 | 0.10 | 199 | a |
| ‘B14528’ | 0.23 | 0.23 | 0.06 | 198 | b |

One of the advantages of the strain ‘RO12756’ is its incompatibility with other commercial strains. Table 10 below provides results of compatibility tests between strain ‘RO12756’ and commercial strain ‘BR06’, commercial strain ‘B14528’, or related strain ‘RO10425’. These results indicate that strain ‘RO12756’ is incompatible with strain ‘BR06’, strain ‘B14528’, and related strain ‘RO10425’. This was true regardless of which tested strain served as the compost variety and which tested strain served as the casing variety.

TABLE 10

| Strains | | | | | | | | |
|---|---|---|---|---|---|---|---|---|
| (compost variety/ | Mean | | Median | | SD | | | Significance |
| casing variety) | lb/ft² | kg/m² | lb/ft² | kg/m² | lb/ft² | kg/m² | Trials | Group |
| ‘RO12756’/‘RO12756’ | 2.62 | 12.79 | 2.61 | 12.74 | 0.41 | 2.00 | 3 | a |
| ‘RO10425’/‘RO10425’ | 2.71 | 13.23 | 2.86 | 13.94 | 0.50 | 2.44 | 3 | a |
| ‘BR06’/‘BR06’ | 2.62 | 12.79 | 2.46 | 11.99 | 1.15 | 5.61 | 2 | a |
| ‘B14528’/‘B14528’ | 2.93 | 14.31 | 2.92 | 14.23 | 0.34 | 1.66 | 1 | a |
| ‘RO12756’/‘BR06’, | 0.70 | 3.42 | 0.62 | 3.03 | 0.25 | 1.22 | 2 | b |
| ‘BR06’/‘RO12756’ | | | | | | | | |
| ‘RO12756’/‘B14528’, | 0.68 | 3.32 | 0.60 | 2.91 | 0.43 | 2.10 | 1 | b |
| ‘B14528’/‘RO12756’ | | | | | | | | |
| ‘RO12756’/‘RO10425’, | 0.91 | 4.44 | 1.04 | 5.08 | 0.35 | 1.71 | 1 | b |
| ‘RO10425’/‘RO12756’ | | | | | | | | |

What is claimed is:

1. A new and distinct variety of Agaricus bisporus Imbach mushroom named ‘RO12756’, as shown and described herein.
